SEQUENCE LISTING (2 sequences)

SEQ ID NO: 1 - DNA, 20 bp, Artificial Sequence
  oligo DNA
  misc_feature (1)..(2): n is a, c, g, t or u
nncactcgggcaccaaggac

SEQ ID NO: 2 - DNA, 100 bp, Artificial Sequence
  damaged DNA
actggggccagatgugtaagccctcccgtatcgtagttatctacacgacggggagtcaggatttcgttcatccatagttgcctgactccccgtcgtgtag

(19) United States
(12) Patent Application Publication
(10) Pub. No.: US 2021/0388417 A1
(43) Pub. Date: Dec. 16, 2021

(54) METHOD FOR REVERSIBLY PROTECTING AND SEPARATING DNA

(71) Applicant: Qufu Normal University, Qufu (CN)

(72) Inventors: Bo CAO, Qufu (CN); Qinghua ZHANG, Qufu (CN); Lili LIU, Qufu (CN); Hong JI, Qufu (CN)

(73) Assignee: Qufu Normal University, Qufu (CN)

(21) Appl. No.: 17/345,583

(22) Filed: Jun. 11, 2021

(30) Foreign Application Priority Data
Jun. 11, 2020 (CN) 202010528082.2

(51) Int. Cl. C12Q 1/6806; C12Q 1/6869

(52) U.S. Cl. CPC C12Q 1/6806; C12Q 1/6869

(57) ABSTRACT

The present disclosure provides a method for reversibly protecting and separation DNA, comprising phosphorylating the 5′-terminal of a target DNA molecule, modifying the 5′-terminal by adenylation; adding adenosine DNA-sensitive exonuclease to samples obtained after termination of the reaction to digest the template; finally, the obtained adenylated modified DNA, that is, the obtained target DNA is separated, and subjected to technical analysis such as sequencing and identification, and the 5′end of the obtained sequence is the site of adenylation modification. The method provided by the present disclosure fills the gap that the prior art cannot accurately locate the break site on genomic DNA, can realize the quantitative and positioning analysis of the break site on DNA samples of different lengths and different sources, and is simple to use and easy to operate. And there are no special requirements for samples, high accuracy, low detection background influence, and high resolution.

### CROSS REFERENCE TO RELATED APPLICATION

This disclosure claims the priority of Chinese Patent Application NO. 202010528082.2 entitled “Method for reversibly protecting and separating DNA” filed with the China National Intellectual Property Administration on Jun. 11, 2020, which is incorporated herein by reference in its entirety.

### TECHNICAL FIELD

The disclosure belongs to the technical field of molecular biology and biomedicine, and specifically relates to a method for reversibly protecting and separating DNA macromolecules.

### BACKGROUND ART

DNA molecules store the genetic information on which organisms rely for survival and reproduction. Therefore, maintaining the integrity of DNA is vital to cells. However, a variety of internal and external environmental factors can lead to biological DNA damage or alteration of the molecules, such as ultraviolet radiation, carcinogenic chemicals, oxidative stress produced during metabolic process of the cells and the like. These damages destroy the integrity of the genome and threaten the stability of the genome. It is now generally accepted that DNA damage is the main cause of cancer and many other aging-related diseases, and thus it is very important to human health.

Among those types of DNA damages, strand break is recognized as one of the most harmful cell damage, because strand break can not only block the processes such as DNA replication, or transcription, but also may lead to recombination events. Therefore, DNA strand break is a research hotspot in the field of life sciences. Among them, studying the location and law of the occurrence of strand breaks in genomic DNA is the foundation for understanding this type of damage.

At present, there are two main techniques to identify the DNA cleavage sites positions within the genome: DSB-Seq, SSB-Seq technique developed by Baranello research group and SSiNGLe technique by Philipp Kapranov research group. In the former technique, biotin and digoxin are used to label the break sites in double strands and the break sites in single strand, respectively, and then the labeled DNA fragments are enriched using an affinity enrichment method and analyzed in combination with next-generation sequencing. During the DNA fragmentation in the SSiNGLe technique, micrococcal nuclease (MNase) is used to digest DNA to produce 3′-terminal phosphate, and DNA cleavage sites having 3′-hydroxyl terminal are labeled and captured by appending a poly-A tail through terminal transferase (TDT), in combination with the next-generation sequencing technique. Although these two methods realize the location of DNA cleavage sites in the whole genome, they have limitations in their application. For example, DSB- and SSB-SEQ has lower resolution for location of the cleavage sites, deeper background during sequencing, while the SSiNGLe technique cannot detect the cleavage at adenine (Adenine. A a) of DNA.

Therefore, in view of the importance of DNA break and the limitations in the current identification technologies, the present disclosure provides a method for reversible protection and separation of target DNA molecules to achieve low-cost, high-sensitivity, and high-resolution location of DNA cleavage sites. And the method of the present disclosure can be further applied to the study of identifying various types of damages and modifications to DNA. This technology will greatly promote the scientific research and clinical application in the fields such as DNA damage-repair process, mechanism of cancer occurrence and cancer prevention, drug safety assessment, gene therapy, and genetic diseases.

### SUMMARY

In view of the problems of deep background, low resolution and low accuracy of existing methods for detecting DNA damages, the present disclosure provides a method for reversible protection and separation of DNA that can be used to detect DNA damages, and the method is capable of locating DNA damage sites with high precision and can be applied to DNA samples of different lengths and sources, the target DNA can be separated and analyzed at single molecule and single nucleoside levels.

In order to achieve the above objective, the present disclosure adopts the following technical solutions.

A method for reversibly protecting and separating DNA, comprising steps of:

(1) subjecting a DNA molecule to enzymatic treatment to obtain a sample containing 5′-phosphorylated DNA;

(2) unwinding the sample containing 5′-phosphorylated DNA to obtain single-stranded DNA;

(3) labeling the single-stranded DNA by 5′-adenylation to obtain a sample containing 5′-adenylated DNA;

(4) digesting the sample obtained in step (3) with adenylation-sensitive 5′-3′ exonuclease, removing the single-stranded DNA that is not modified by 5′-adenylation, and purifying the sample to obtain a target DNA molecule that is modified by 5′-adenylation;

(5) subjecting the target DNA molecule with adenylation modification to deadenylation treatment to obtain a target DNA molecule.

In step (1), there is no limitation on the source of the DNA molecule, which can be artificially synthesized or extracted from animals, plants or microorganisms. Common methods for DNA extraction in the art may be used based on different samples, such as phenol extraction method, isopropanol precipitation method, CTAB method, etc. Furthermore, commercial kits may be used.

In step (1), the DNA molecule may be double-stranded or single-stranded. Optionally, when the DNA molecule is single-stranded, step (2) may be omitted.

In step (1), the enzyme is an enzyme that is capable of converting 5′-hydroxyl DNA into 5′-phosphorylated DNA such as, T4 polynucleotide kinase. The enzyme may also be an excision repair enzyme involved in DNA damage sites. These enzymes are capable of producing the 5′-phosphate-terminals such as DNA glycosylase and endonuclease. One or more of the DNA glycosylases such as uracil DNA glycosylase (UDG), 8-oxoguanine DNA glycoside enzyme (hOGGl), formamide pyrimidine DNA glycosylase (FPG), thymine DNA glycosylase (TDG) and endonuclease IV are selected based on to the specific fragments to be detected.

Preferably, in step (2), the unwinding process is thermal denaturation. Specifically, the step of obtaining single-stranded DNA includes: placing the DNA on ice immediately after thermal denaturation to maintain the single-stranded state.

In step (3), labeling 5′-adenylation comprises modifying the DNA 5′-terminal by an enzyme. The enzyme is a common DNA adenylation enzyme such as Mth RNA Ligase, or T4 DNA Ligase. The DNA adenylation enzyme modifies 5′-terminal of the DNA by adenylation in the presence of ATP. In the embodiments of the present disclosure, a kit containing Mth RNA Ligase and ATP is used for reaction.

In step (4), the adenylation-sensitive 5′-3′ exonuclease includes, but is not limited to, any adenylation-sensitive exonucleases such as T5 exonuclease, or RecJ exonuclease.

Preferably, steps (1), (3), and (5) each further comprise purification of the sample after reaction.

The above method can be used to detect modifications and damages of the DNA molecules.

A method for detecting damage and modification sites in a DNA molecule by using the above method, comprising:

(i) extracting a DNA molecule, disrupting and dephosphorylating the DNA molecule to obtain a sample;

(ii) obtaining a target DNA molecule by using the methods according to the above method for reversibly protecting and separating DNA

(iii) sequencing the target DNA molecule and performing analysis and alignment to obtain the damage or modification sites.

In step (i), disrupting is performed by sonication, with a fragment size of preferably 200-500 bp.

In step (iii), sequencing includes, but not limited to Sanger sequencing, Illumina sequencing. Samples containing single site is subjected to Sanger sequencing, and samples containing multiple sample sites are subjected to Illumina sequencing. Preferably, when Illumina sequencing is used in step (iii), step (iii) further comprises the following steps: performing PCR amplification after the target DNA molecule is converted into double-stranded DNA and the product of PCR amplification is used for Illumina sequencing.

The principle for the method of present disclosure is as follows.

Firstly, after DNA sample is thermally denatured, target DNA molecules are subjected to 5′-terminal phosphate treatment, and phosphated 5′-terminal of the DNA is subjected to reversible modification through adenylation by using adenyltransferase, thereby protecting the target DNA molecules. Secondly, the unprotected DNA in the sample is digested with adenylation-sensitive 5′-3′ exonuclease, and the DNA with 5′-terminal modified by adenylation is resistant to hydrolysis by nuclease and is thus retained, DNA with 5′-terminal modified by adenylation after digestion is purified to eliminate the influence of the background. Thirdly, the adenylation is removed by eliminating the enzyme adenylation modification at 5′-terminal with deadenylase to revert to the phosphorylation modification at 5′-terminal, thus achieving reversible protection and separation of target molecules. Finally, DNA with adenylation modification at 5′-terminal being removed is sequenced, and the 5′-terminal obtained by the sequencing is the original protected site, i.e., the DNA cleavage site.

The present disclosure has the following advantages.

The present disclosure provides a method for capturing and separating target DNA fragments. In the method, reversible protection of 5′-terminal is used for quantitative analysis and positioning for DNA of different lengths and different sources, which is easy to use and operate. Meanwhile, there are special requirements for the samples, and high accuracy is ensured. The method of the present disclosure is less effected by detection background and has high resolution, which can be widely used in molecular diagnosis, safety assessment of chemotherapeutic drugs, cancer occurrence and prevention, molecular biology research, gene therapy and many other fields.

### BRIEFT DESCRIPTION OF THE DRAWINGS

FIG. 1 is a schematic diagram of the flow chart of reversible protection and separation of DNA.

FIG. 2 is a diagram showing the results for adenylation modification of a sample of short-stranded DNA.

FIG. 3 is a diagram showing the results for hydrolysis resistance to adenylated DNA.

FIG. 4 is diagram showing the results for deadenylation of adenylated DNA.

FIG. 5 is a diagram showing the detection results for precisely locating the damage sites of short-strand DNA.

FIG. 6 is a diagram showing the detection results for precisely locating the damage sites of genomic DNA.

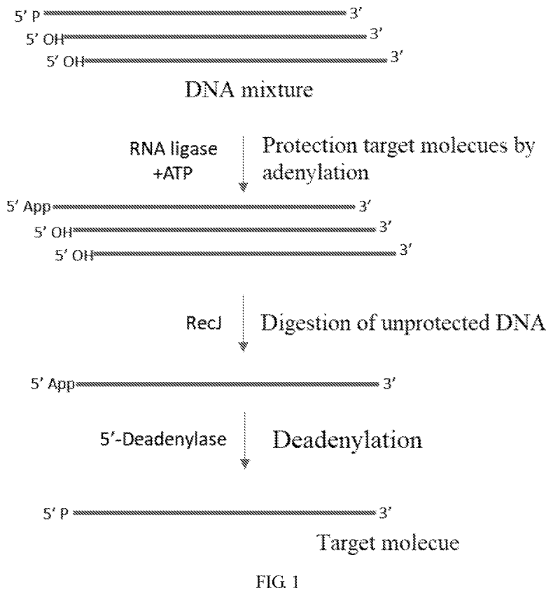

### DETAILED DESCRIPTION OF THE EMBODIMENTS

The present disclosure will be further described below in conjunction with the embodiments and drawings, but the present disclosure is not limited by the following embodiments.

### Example 1

DNA modification by adenylation and analysis of its anti-nuclease activity

A DNA single-stranded fragment (20 nt, SEQ ID NO. 1: 5′Phos/-NNCAC TCG GGC ACC AAG GAC-3′) containing a modification of 5′-phosph and a Homeobox DNA without phosphate modification were synthesized by a DNA synthesis company (IDT, USA).

The short DNA fragments with phosphorylation modification and without modification were mixed in equal proportion and used as a DNA substrate. The 20 μL reaction system contains 100 pmol of Mth RNA Ligase (NEB, USA), 2 μL of 1 mM ATP (NEB. USA), 2 μL of DNA Adenylation Buffer (NEB, USA), 2 μg of DNA substrate, and the reaction system was made up to 20 μL with water. After reacting at 65° C. for 1.5 h, the reaction system was inactivated at 85° C. for 5 min.

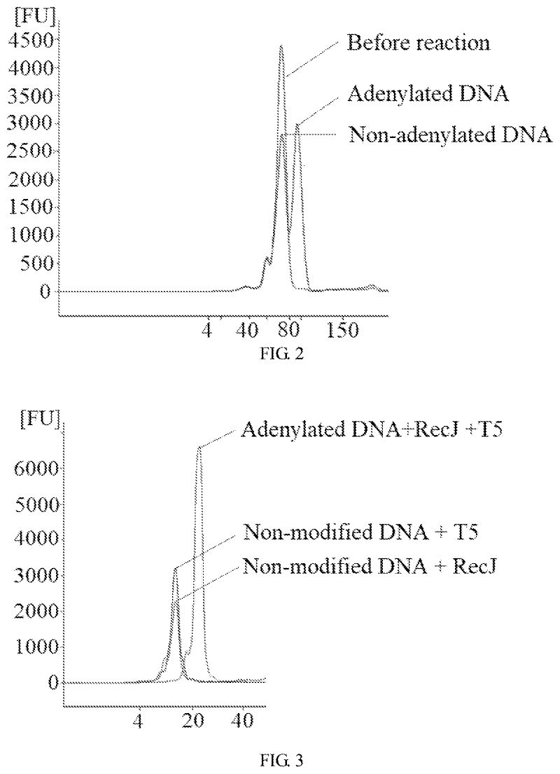

The reaction product was purified with a DyeEx DNA Purification Kit 2.0 spin kit (QIAGEN) and analyzed by Bioanalyzer (Agilent) Small RNA Chip. The results are shown in FIG. 2. As shown in FIG. 2, before the reaction, phosphorylation-modified DNA and non-modified DNA cannot be separated during electrophoresis in Bioanalyzer, forming a peak (corresponding to a band). After the reaction, DNA containing phosphorylation modification is modified by adenylation, and the electrophoresis slowed down during electrophoretic analysis in Bioanalyzer, allowing separation from DNA without adenylation modification.

DNA with 5′-adenylation modification and DNA without modification were subjected to analysis of hydrolysis by RecJ and T5 exonuclease, respectively. In a 20 μL reaction system, containing 1 μg of DNA substrate, 10 units of exonuclease T5 (NEB) or 30 units of exonuclease RecJf (NEB), reaction was carried out at 37° C. for 1.5 hours, and inactivated at 65° C. for 20 minutes. The reaction product was purified with DyeEx DNA purification kit 2.0 spin kit (QIAGEN), and then analyzed using Bioanalyzer (Agilent) Small RNA Chip. The results are shown in FIG. 3 below. In FIG. 3, DNA with adenylation modification can resist hydrolysis by exonucleases of RecJ and T5, and DNA without adenylation modification may be hydrolyzed by RecJ exonuclease or T5 exonuclease.

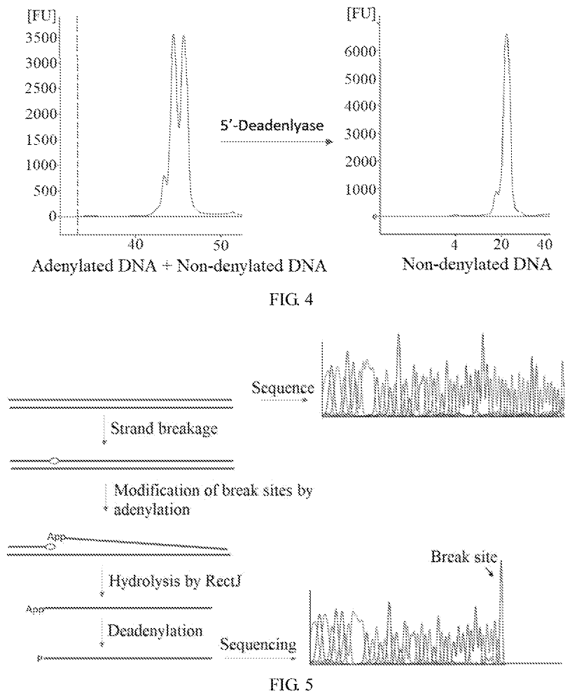

Reversible removal of adenylation modification: adenylated DNA (5′ App DNA) was reverted to its initial state (5′-phosphorylation-modified DNA) with Deadenylation Kit (NEB). 20 μL of reaction system containing 50 units of deadenylation enzyme 5′-Deadenylase (NEB, USA), 2 μL of buffer (NEB Buffer1), 50 ng of adenylation-modified short-chain DNA substrate, 50 ng of short chain DNA substrate without modification was made up with water to 20 μL. Reaction was carried out at 30° C. for 1 hour, and inactivated at 70° C. for 20 minutes. The reaction product was purified with DyeEx DNA Purification Kit 2.0 spin kit (QIAGEN) and analyzed by Bioanalyzer (Agilent) Small RNA Chip. The result is shown in FIG. 4. Before the reaction, the adenylation-modified DNAs, which were mixed with the phosphorylation-modified DNA in equal proportion, were all deadenylated into phosphorylation-modified DNA after the reaction, thus realizing reversible reaction of the DNA adenylation.

### Example 2

Application of technology of reversible protection and separation by adenylation in identification of DNA damage sites

1. AP Site

(1) by DNA synthesis companies (the IDT Corporation, USA) containing synthetic 100 bp of DNA double-stranded fragments, sense strand sequence:

SEQ ID NO. 2:
ACTGGGGCCAGATGUGTAAGCCCTCCCGTATCGTAGTTATCTACACGACG
GGGAGTCAGGATTTCGTTCATCCATAGTTGCCTGACTCCCCGTCGTGTA
G,

underlined is the DNA damage (Uracil) site;

(2) Enzyme digestion of DNA damage sites: A 50 μL reaction system containing 10 units of Uracil repair enzyme UDG and endonuclease IV (NEB, USA), 5 μL of buffer (NEB Cutsmart Buffer), 1 μg of DNA substrate was made up to 50 μL with water. After reacting at 37° C. for 1 h, the reaction system was inactivated at 75° C. for 20 min;

(3) DNA denaturation: DNA sample was placed in a PCR machine, and the sample was quickly placed on ice after thermal denaturation at 95° C. for 3 minutes;

(4) Reversible labeling of break points: 20 μL×2 systems containing 100 pmol of Mth RNA Ligase (NEB, USA), 2 μL of 1 mM ATP (NEB, USA), 2 μL of DNA Adenylation Buffer (NEB, USA), 2 μg of 5′-phosphorylated DNA substrate were made up to 20 μL with water. After reacting at 65° C. for 1.5 h, the reaction system was inactivated at 85° C. for 5 min.

(5) Purification of the labeled DNA fragments: Using a Zymo DNA purification kit, 100 μL of binding solution and 400 μL of absolute ethanol were added to the reaction product, mixed thoroughly and passed through a column, rinsed once with 750 μL of wash buffer, and eluted with 20 μL of eluent.

(6) Elimination of the template: 10 units of exonuclease enzyme T5 (NEB) or 30 units of exonuclease enzyme RecJf (NEB) was added to the purified solution, reacted at 37° C. 1.5 h, and inactivated at 65° C. for 20 min. The reaction product was just the isolated adenylation-modified DNA, which was purified using DyeEx the DNA purification kit 2.0 Spin kit (QIAGEN) and would be used for the reaction in step (7).

(7) Removal of breakpoint label: adenylated DNA (5′App DNA) was reverted to initial state (5′p-DNA) using Deadenylation Kit (NEB). A 20 μL reaction system containing 50 units of deadenylation enzyme 5′-Deadenylase (NEB, USA), 2 μL of buffer (NEB Buffer1), and short-chain DNA substrate obtained in step (6) was made up to 20 μL with water. Reaction was carried out at 30° C. for 1 h and inactivated at 70° C. for 20 min.

The product obtained in step (7) was sent to a sequencing company (Genewiz) for Sanger sequencing. The results are shown in FIG. 5, and 5′-terminal obtained by sequencing is the original damage site.

2. Detection of Oxidative Damage Sites in Escherichia coli

(1) An Escherichia coli strain of DH10B was cultured in 10 mL LB culture at 37° C. till OD600=0.5, and the culture was placed on ice for 20 min, then 0.2 mM hydrogen peroxide was added for treatment for 30 min. 1 mL of bacterial cells was collected and extracted for genomic DNA with OMEGA Bacterial DNA Kit (OMEGA, USA). The extraction method was conducted in accordance with the product instructions.

(2) 5 μg of extracted genomic DNA was taken and added, and made up to 100 μL with ultrapure water, and an ultrasonic breaker was used to fragment the DNA into fragments of about 500 bp.

(3) 26 μL of the DNA obtained in step (2) was treated by dephosphorylation: 3 μL of Cutsmart Buffer (NEB) and 1 μL Shrimp Alkaline Phosphatase (rSAP, NEB) were added, reacted at 37° C. for 30 min, and inactivated at 70° C. 10 min.

(4) Restriction enzyme digestion of the DNA damage sites: 50 μL of reaction system containing 10 units of Uracil repair enzyme UDG and endonuclease IV (NEB, USA), 5 μL of buffer (NEB Cutsmart Buffer), and 1 μg of DNA substrate was made up to 50 μL with water. After reacting at 37° C. for 1 hour, the reaction system was inactivated at 75° C. for 20 minutes. The reaction product was purified with DyeEx DNA Purification Kit 2.0 spin kit (QIAGEN).

(5) DNA denaturation: the DNA sample obtained in step (4) was placed in a PCR machine, and quickly placed the sample on ice after thermal denaturation at 95° C. for 3 minutes.

(6) Reversible labeling of breakpoints: 5′-phosphorylated terminal was converted to adenylation modification by using 5′-DNA adenylation kit (NEB, USA). A reaction system contained 2 μL of Mth RNA Ligase, 2 μL of 1 mM ATP, 2 μL of DNA Adenylation Buffer, and the DNA substrate obtained in step (5) was made up to 20 μL with water. The reaction was carried out at 65° C. for 1.5 h and then inactivated at 85° C. to 5 min;

(7) Elimination of the templates: 10 units of exonuclease endonuclease T5 (NEB, USA) or 30 units of exonuclease RecJf (NEB, USA) was added to the purified solution, reacted at 37° C. for 1.5 h and inactivated at 65° C. for 20 min. Control: Non-adenylated DNA was digested with the same system.

(8) Purification of labeled DNA fragments: using Zymo DNA purification kit, 100 μL of binding solution and 400 μL of absolute ethanol was added to the reaction product, mixed thoroughly and passed through a column. The column was rinsed once with 750 μL of wash buffer, and then eluted with 10 μL of eluting agent.

(9) Removal of breakpoint label: deadenylation was conducted using Deadenylation Kit (NEB, USA). A reaction system containing 50 units of deadenylation enzyme (NEB, USA), 2 μL of buffer (NEB Buffer1), and DNA substrate obtained in step (6) was made up to 20 L with water. The reaction was carried out at 30° C. for 1 h and inactivated at 70° C. for 20 min. The reaction product is purified according to the method in step (8).

(10) Construction of Illumina library: DNA library was constructed with the eluted DNA obtained in step (9), using the Clontech SMART ChIP-seq kit for. Kit instructions were followed: 1 mM of adaptor DNA and MML viral reverse transcriptase were added and reacted at 50° C. for 2 hours. The reaction was terminated at 70° C. for 10 min. Then PCR amplification was performed under the conditions of denaturation at 95° C. for 30 s, annealing at 50° C. for 30 s, and extension at 68° C. for 30 s, 15 cycles. The product was sent to the sequencing company for Illumina sequencing.

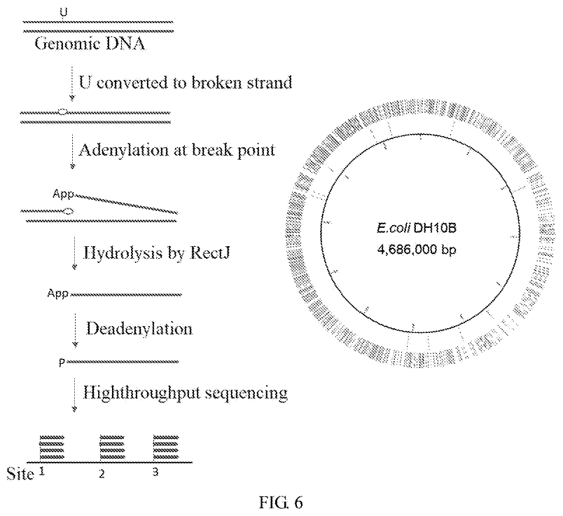

(11) Analysis of Illumina sequencing data: the obtained sequencing data were matched to E. coli genome, and 5′-terminal in the region where the reads were concentrated is shown in FIG. 6, which is the DNA damage site.

1. A method for reversibly protecting and separating DNA, comprising steps of:
(1) subjecting a DNA molecule to enzymatic treatment to obtain a sample containing 5′-phosphorylated DNA;
(2) unwinding the sample containing 5′-phosphorylated DNA to obtain single-stranded DNA;
(3) labeling the single-stranded DNA by 5′-adenylation to obtain a sample containing 5′-adenylated DNA;
(4) digesting the sample obtained in step (3) with adenylation-sensitive 5′-3′ exonuclease, removing the single-stranded DNA that is not modified by 5′-adenylation, and purifying the sample to obtain a target DNA molecule that is modified by 5′-adenylation;
(5) subjecting the target DNA molecule with adenylation modification to deadenylation treatment to obtain a target DNA molecule.

2. The method according to claim 1, wherein in step (1), the DNA molecule is double-stranded or single-stranded, and when the DNA molecule is single-stranded, step (2) is omitted.

3. The method according to claim 1, wherein in step (1) the enzyme is an enzyme that is capable of converting 5′-hydroxyl DNA into 5′-phosphorylated DNA, and the enzyme is selected from T4 polynucleotide kinase and an excision repair enzyme targeting DNA damage sites;
and wherein in step (4), the adenylation-sensitive 5′-3′ exonuclease is selected from T5 exonuclease, RecJ exonuclease, and combination thereof.

4. The method according to claim 1, wherein in step (2), unwinding the sample containing 5′-phosphorylated DNA comprises thermal denaturation.

5. The method according to claim 1, wherein in steps (1), (3), and (5), further comprising purifying the sample after reaction.

6. A method for detecting damage and modification sites in a DNA molecule by using the method according to claim 1, comprising:
(i) extracting a DNA molecule, disrupting and dephosphorylating the DNA molecule to obtain a sample;
(ii) obtaining a target DNA molecule by using the methods according to claim 1; and
(iii) sequencing the target DNA molecule and performing analysis and alignment to obtain the damage or modification sites.

7. The method according to claim 6, wherein the step (i) disrupting is performed by sonication, with a fragment size of preferably 200-500 bp.

8. The method according to claim 6, wherein in step (iii), sequencing is performed by Sanger sequencing or Illumina sequencing.

9. The method according to claim 8, wherein in step (iii), sequencing is performed by Illumina sequencing, and the method further comprises a step of PCR amplification after the target DNA molecule is converted into double-stranded DNA, and the product of PCR amplification is used for Illumina sequencing.
